(19) United States
(12) Patent Application Publication
(10) Pub. No.: US 2004/0147973 A1
(43) Pub. Date: Jul. 29, 2004

(54) Intra cardiac pacer and method

(76) Inventor: Robert G. Hauser, Long Lake, MN (US)

Correspondence Address: Beck & Tysver, P.L.L.C., Suite 100, 2900 Thomas Avenue S., Minneapolis, MN, 55416, US

(21) Appl. No.: 10/606,491

(22) Filed: Jun. 26, 2003

Related U.S. Application Data
(60) Provisional application No. 60/392,188, filed on Jun. 27, 2002.

(51) Int. Cl. A61N 1/375

(52) U.S. Cl. 607/36

(57) ABSTRACT

An intracavitary pacemaker for implantation into a cardiac chamber or a portion of a cardiac chamber such as the left atrial appendage is disclosed. Both therapeutic and diagnostic pacing algorithms are available from the intracavitary electrode sites.

### CROSS REFERENCE

This application claims the benefit of provisional patent application 60/392,188 filed Jun. 27, 2002 which is incorporated in its entirety herein.

### FIELD OF THE INVENTION

The present invention relates generally to pacing and more particularly to an implantable pulse generator for the management of a variety of heart disorders.

### BACKGROUND OF THE INVENTION

Pacing or pacemaking is a very well accepted therapy for bradycardia and congestive heart failure. Atrial tachyarrhythmia and other complex rhythm disturbances have been candidates for pacing therapy, however reliable stimulation therapy is difficult to define for the patient. For example, in some patients an atrial anti-tachyarrhythmia pacing therapy is effective and well tolerated while in other instances a similar therapy for a similar tachyarrhythmia can be ineffective. There is an unmet need for improved atrial pacing therapies.

In general, pacing devices monitor the presence or absence of a cardiac depolarization within a defined time interval and this information is used to guide the stimulus therapy. In most instances the information and therapy are dispensed based primarily upon sensed heart rate. That is, pacers cannot distinguish tachyarrhythmia except based on observed rate, measured over several heartbeats. There is an unmet need to develop non-rate based technique for detecting atrial arrhythmias.

Initially implanted pacers (IPGs) incorporated integral pacing leads, which were sutured directly to the epicardial surface of the heart in an open procedure. Later transvenous pacemaker leads were developed and IPG s were separated from the lead system. In these instances the IPG could be replaced without disturbing the lead system. At about this time, as an alternative, an intra cavity pacer was proposed which was “leadless” and wholly implanted within a chamber of the heart. See for example U.S. Pat. No. 3,943,936 to Rasor et al. These devices were battery powered or “self powered” and they never reached commercialization. There is an unmet need for a reliable intra cardiac pacer.

### SUMMARY OF THE INVENTION

By way of contrast, the present invention teaches the use of one or more implantable intra cavitary or intra cardiac pulse generators (ICP) that can operate alone or form a cluster or network of semi-autonomous devices. These devices cooperate to better interpret the origin and progression of arrhythmias. The ICP can intervene alone to treat pathologic rhythms or it may operate in concert with other implanted devices to deliver or enhance a therapy.

In certain instances the ICP device can invoke a therapy from a companion device such as an Implantable Cardioverter Defibrillator (ICD) or other pacemaker or Implanted Pulse Generator (IPG).

The size and shape of the ICP device facilitates implantation in the right atrial appendage (RAA) or the left atrial appendage (LAA). The device may also be conveniently placed in the right ventricle (RV) or left ventricle (LV). Versions of the device may also be implanted into the right atrium (RA) or the left atrium (LA). Depending on the number of devices and the locations of the devices the group can deliver conventional pacing modalities or unconventional pacing modalities. The ICP may have an attached lead system that couples the device to another device or chamber of the heart remote from the chamber of ICP implantation. The additional ICP lead may be passed intra-cardially or epicardially from the ICP implantation site.

The volume of the ICP is small and it is adapted to its implantation site by the use of a shroud or shield that surrounds and encapsulates the ICP anchoring the ICP to an appropriate anatomic structure. The shield for implantation in the LAA differs from the shield for placement in an open chamber. In most instances the shield acts as an anchoring device and it also includes electrical contacts or electrodes so that all or a portion of the shield acts as a pacing electrode for sensing and/or pacing.

Communication between the ICP and other implanted or external devices may be accomplished through radio frequency (RF) or sub threshold current pulses delivered through the body of the patient. In one embodiment the ICP is connected to an implantable cardioverter defibrillator (ICD) though a simplified defibrillation lead system. Acoustic signaling is a possibility as well.

A conventional battery such as a solid-state lithium cell or a rechargeable cell may supply the power for the ICP device. The circuitry of the ICP is of conventional construction as well known in this industry and need not be described in detail.

It is anticipated that a tether or catheter/stylet will be temporarily be coupled to the device for manipulating the device during implantation. Once secured, the tether may be released and the catheter removed from the heart. In some embodiments the tether itself remains attached to the ICP and it may be used as a power supply recharging wire or used to guide other interventional devices to the location of the ICP.

The stimulation regime provided by or commanded by the ICP is described throughout as encompassing pacing or defibrillation energy levels. However these examples are selected to provide clarity in the description. It must be understood that the typical ICP implantation site allows other non-traditional stimulation levels to be delivered therapeutically. For example, a multiphasic current pulse can be used to modify the calcium channels of the heart tissue to improve contractility. These multiphasic current pulses are best delivered from spaced apart electrode site such as those taught by several embodiments of this invention. The magnitude of these pulses is typically 7 volts or about 20 minutes in duration. Thus the theory lies between the energy used for pacing and the energy levels used for defibrillation.

### BRIEF DESCRIPTION OF THE DRAWINGS

Throughout the figures identical reference numerals identify identical or equivalent structure, wherein:

FIG. 1 is a schematic representation of a representative hardware implementation;

FIG. 2 is a schematic representation of a representative hardware implementation;

FIG. 3 is a schematic representation of a representative hardware implementation;

FIG. 4 is a schematic representation of a representative hardware implementation;

FIG. 5 is a schematic representation of a representative hardware implementation;

FIG. 6 is a schematic representation of a representative hardware implementation;

FIG. 7 is a schematic representation of a representative hardware implementation;

FIG. 8 is a schematic representation of a representative hardware implementation;

FIG. 9 is a schematic representation of an implantation configuration;

FIG. 10 is a schematic representation of an implantation configuration;

FIG. 11 is a schematic representation of an implantation configuration;

FIG. 12 is a schematic representation of an implantation configuration;

FIG. 13 is a schematic representation of an implantation configuration;

FIG. 14 is a schematic representation of an implantation configuration;

FIG. 15 is a schematic representation of an implantation configuration;

FIG. 16 is a timing diagram showing a cardiac rhythm treatment using the ICP in the LAA;

FIG. 17 is a timing diagram showing a cardiac rhythm treatment using the ICP in the LAA; and

FIG. 18 is a schematic representation of an interconnected ICD and ICP.

### DETAILED DESCRIPTION

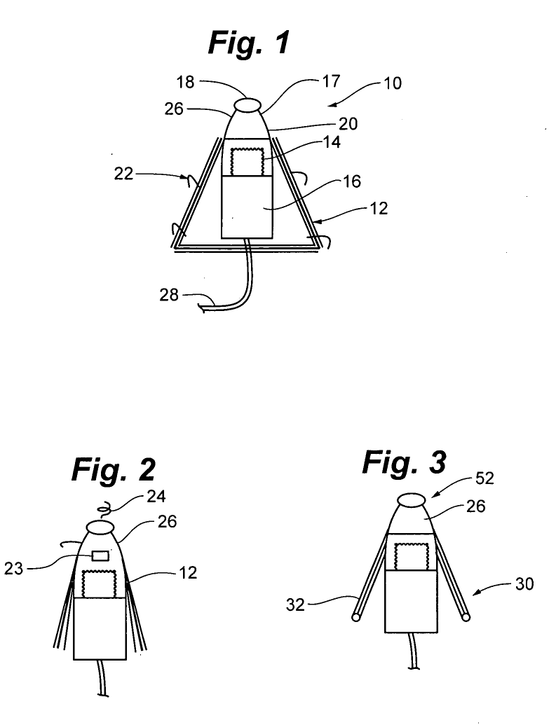

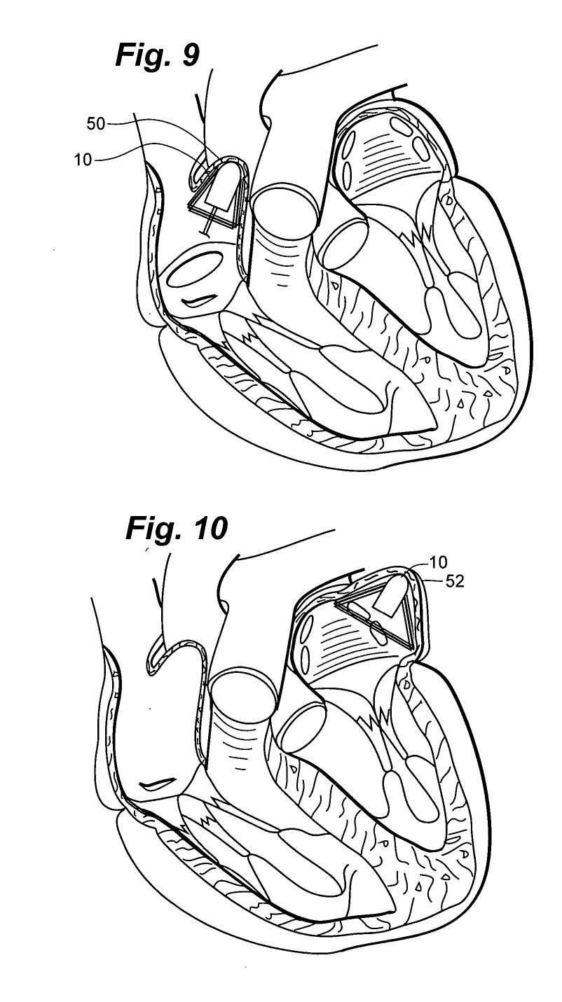

To facilitate description of the invention the text is partitioned into a first section directed to the hardware implementation of a typical ICP device in connections with FIG. 1 through FIG. 8 and FIG. 18. Secondly a description of representative therapy modes, methods and configurations are set forth in connection with FIG. 9 though FIG. 17.

It should be understood that the description is directed to representative implementations and additional configurations and modes of operation are within the scope of the invention.

Hardware Implementation

FIG. 1 is a schematic diagram showing a typical implementation of the ICP 10 and its associated and complementary shield 12. The hermetically sealed housing 17 of the ICP 10 is seen in partial cut away showing the location of the electronic circuitry 14, the relatively large output capacitor 15 and the battery 16 energy source. In this configuration the metal housing 17 of the ICP 10 is partitioned into a cathode electrode 18 at the most distal tip of the housing and an anode electrode pole 20 on the housing 17. The housing itself may form the anode pole. This drawing shows the shield 12 in its fully deployed state. In this configuration the shield is resilient and expands to conform to the shape of the anatomic structure that is used to retain the ICP. Optional hooks typified by the representative hook 22 may be incorporated in to the shield design to assist in anchoring the ICP. The particular shield design depicted is intended for an appendage implantation. An umbilical wire 28 may be provided to assist in placement of the device 10 and in some embodiments to recharge the battery 16 of the device 10. This tether is also a safety feature in that it can be used to manipulate and guide other deployment and retrieval devices to the implantation site.

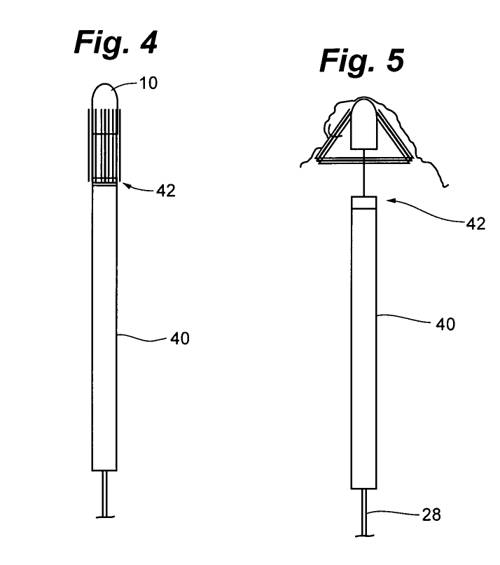

FIG. 2 shows an alternate fixation strategy where a helical screw tip 24 projects from the housing 26 to form the cathode pole for sensing and pacing. It is anticipated that the stylet (see FIG. 4 or 5) will be used to rotate the entire device to screw in the electrode. Alternatively, the stylet may transfer torque through a movable screw to advance the electrode into tissues. It is expected that this configuration will allow the device 10 to be safely anchored in tissues like the left or right atrial appendage. Sensor 23 can be incorporated into the device to accommodate activity based pacing modalities. The heart motion may be filtered out or used as an independent indicator of heart function.

FIG. 3 shows an alternate fixation strategy for use in the right or left ventricular chambers. In this instance the shield takes the form of tines 30 and 32 to engage the trabeculae of the chamber.

FIG. 4 and FIG. 5 should be considered together. FIG. 4 shows a catheter 40 used to deploy the device coupled to the device 10. The distal tip of the deployment catheter 42 captures the shield 12 and holds it in a collapsed configuration to permit it to be navigated through the vasculature. In this embodiment the catheter 40 may act as the stylet or it may act as a deployment device, or both.

FIG. 5 shows the catheter retracted releasing and deploying the device 10 into an atrial appendage. The umbilical wire in this implementation acts as a safety wire allowing the catheter to be easily reconnected to the device 10 to permit acute repositioning.

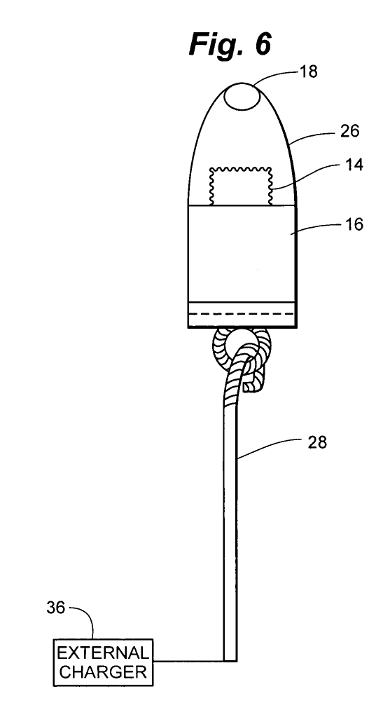

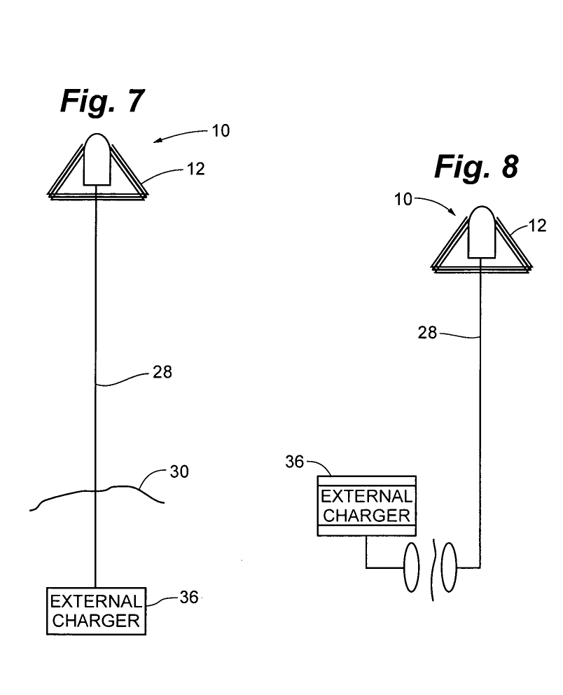

FIG. 6 shows an alternate use of the umbilical wire 28. In this embodiment the wire can be used to recharge the battery 16. In general the umbilical wire 28 will be coupled to a remote and exterior charging system 36. The umbilical wire 28 may be exteriorized across the skin in a percutaneous manner as seen in FIG. 7 or the umbilical 28 may be used as one component of a trans-cutaneous recharging system 36 as seen in FIG. 8.

Thus the preferred mechanical and electrical partitioning of the ICP device has been presented. It should be apparent that other partitioning of the structure might be carried out without departing from the scope of the invention.

Configuration and Operation

FIG. 9 is a schematic representation of a simple application of a single ICP 10 implanted to treat a patient. In the figure the ICP 10 is placed in the right atrial appendage RAA 50. The ICP 10 would have pacing parameters appropriate to single chamber atrial pacing modes including AAI, AAT and AOO.

In this single chamber approach anti tachy modalities can be used including Burst, Scanning Burst, and Overdrive pacing. These modalities are widely known and do not require further description. Although the location in the right heart is conventional for pacing therapy, the atrial appendage is an unusual site for sensing and pacing. It is expected that the delivery of stimulation from this location will be effective and the absence of lead body will be a benefit to the patient. It is also important to recognize that sensing in the RAA or LA differs from conventional atrial placement of a lead. For example the SA node, which imitates the depolarization, is “close by” and it is expected that the activation sequence of the heart will be apparent from comparisons between the sense events on atrially placed leads in comparison with sense events detected in the LAA or RAA or both. Knowledge of the activation sequence can be used to guide therapy on a beat-by-beat basis.

FIG. 10 shows a schematic representation of a single chamber application with the ICP device 10 placed in the left atrial appendage LAA 52. The implantation site is preferred because it minimizes the creation of thrombus in the event of a prolonged episode of atrial tachy arrhythmia or flutter or fibrillation. Once again conventional atrial pacing modalities are available from this location. The physical placement of the device 10 in the LAA provides some therapeutic effects without regard to the pacing modalities selected for the device. For example, this location cannot generate clots if it is plugged with an ICP.

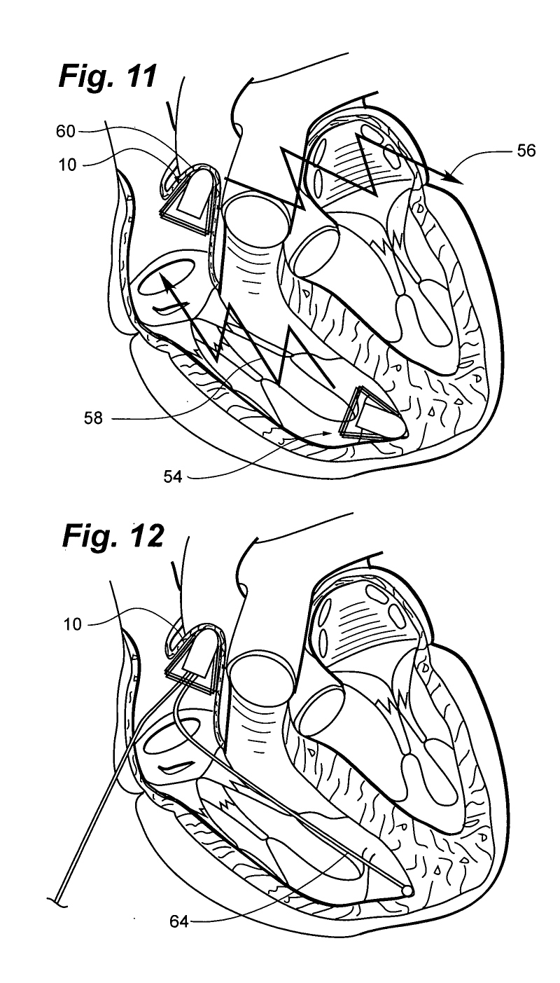

FIG. 11 is a schematic representation of a dual chamber pacing modality carried out with two ICPs networked together. The figure shows how a ventricular ICP 54 of the type seen in FIG. 3 may be lodged in the apex of the ventricle of a patient. At the same time an ICP 10 of the type depicted in FIG. 1 or 2 is lodged in the right atrium 60 of the patient. In this dual chamber mode the devices will communicate with each other as depicted by the communication arrows 56 and communication arrow 58. In this configuration the two devices can cooperate to provide dual chamber pacing modalities including but not limited to DDD, VDD, and DVI modalities. In general, it is desirable but not essential that the two devices communicate with each other. Alternative bidirectional communication links can be provided as well, with acoustic triggered stimuli and sub-pacing electrical stimuli included within the depiction 56 and 58. Each device will broadcast the occurrence of sense and pace events to other near by devices. The communication will be encoded to reflect the location and pacing modality programmed into the device. The timing events will be broadcast on a near real time basis.

FIG. 12 shows an alternative method of carrying out dual chamber pacing using a design where a ventricular lead 64 descends from the ICP 10 to provide sensing and pacing electrode sites in a ventricle. The figure should be understood to encompass the use of an ICP in the left or right atrium or in the left or right atrial appendage with the ventricular lead in either the left or right ventricle. In this embodiment the device does not need the elaborate communication schema set forth in FIG. 11. All conventional dual chamber modalities are available with this configuration including but not limited to DDD, DVI and VAT and VDD.

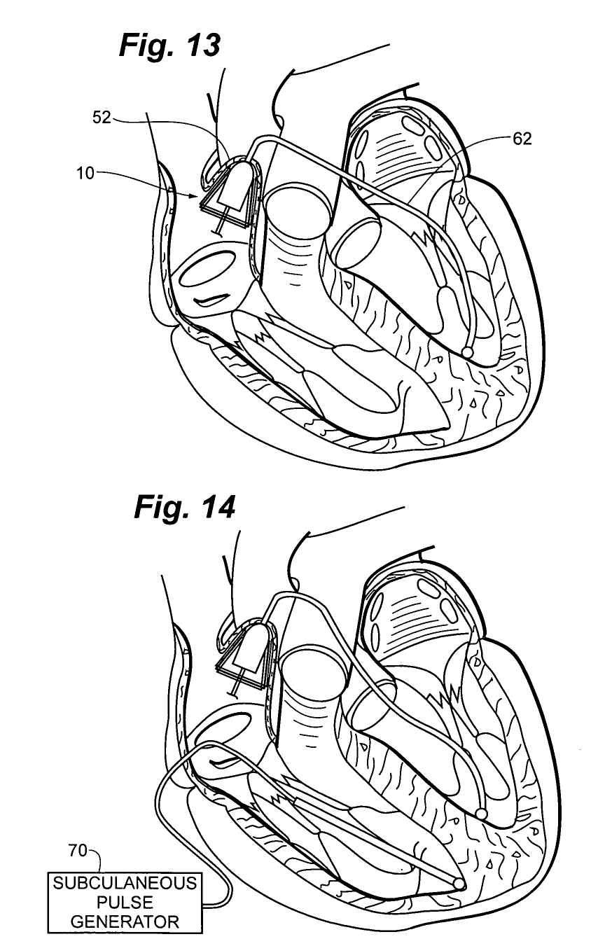

FIG. 13 represents a preferred configuration where the ICP 10 is implanted in the left atrial appendage 52 (LAA) and the ventricular lead exits from the appendage and is routed on the outside of the heart to an epicardial location on the left ventricle. In this configuration the ICP will have the distal tip configured to accept a lead body with electrical coupling to the electrode sites located on the epicardial surface of the heart. This configuration allows the ICP to block the LAA in the event of atrial fibrillation. By routing the ventricular lead outside of the heart it is anticipated that the impact on clotting will be minimal. In this example an epicardial lead 62 is connected to the left ventricle. This lead is coupled to the ICP in the LAA and it is routed through the wall of the LAA to the space outside the heart. It is expected that this configuration will be well suited to patients with atrial tachycardia and incipient congestive heart failure. By routing the lead 62 out side the heart thrombus complications are eliminated and any suitable location on the left ventricle is available for lead placement. All conventional pacing modalities can be delivered by this configuration including ventricular resynchronization therapies. There are two distinct benefits for this configuration. First the LAA is blocked preventing thrombus formation. Secondly the epicardial placement is unrestricted and sites not reached by a coronary sinus lead are available to optimize the resynchronization therapy. As an alternative it should be understood that the ICP can be implanted in the RAA and the ventricular pacing lead guided though the interior of the heart to LV. In summary the appendages can be used to hold the device while the ventricular electrode location is reached by tunneling the lead outside the heart.

FIG. 14 shows an alternative ICP 10 located in the RAA 50 with a ventricular lead 66 exiting the device and entering the coronary sinus. In this configuration the ICP 10 provides ventricular stimulation alone or in concert with a right ventricular stimulation device 70 or biventricular ICD. This is a preferred configuration for bi-ventricular pacing. In this instance the left and right ventricles have leads attached. The stimulation regime may readily shorten the QRS complex by appropriate setting of the ventricular stimulation parameters.

In this configuration the ICP provided right atrial and left ventricular pacing and sensing while the implanted conventional pacer 70 or biventricular ICD provides ventricular pacing to the right heart. Communication between the implanted ICP and the ICD/IPG can coordinate the delivery of pacing therapies to “narrow” the QRS times. This form of treatment for “wide” QRS syndromes is associated with congestive heart failure. FIG. 14 should also be understood to encompass the coordination of the ICP with an implanted ICD that is normally associated with a ventricular lead placement in the right heart.

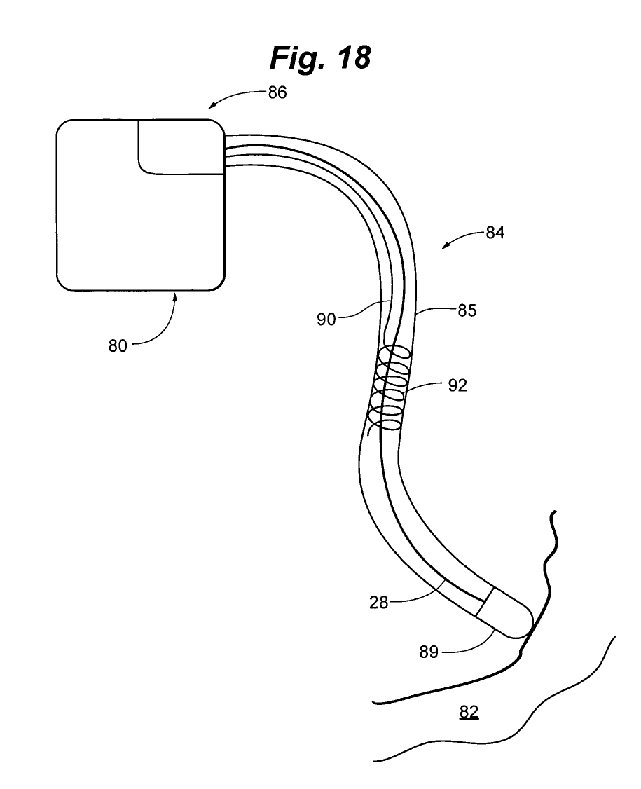

FIG. 18 shows a conventional ICD or IPG 80 coupled to a distal ICP 89 implanted in heart tissue 82 in a ventricular chamber. This strategy allows for a simplified lead system 84 that has three connections to the proximal connector 86 of the ICD/IPG 80. Inside the insulative lead body 85 there is a coil wire 90 is which emerges from the lead body and forms a large area electrode 92, separately terminated at the connector 86. A separate umbilical wire 28 for communicating with the ICP 89 is carried within the lead body 85. In this configuration the simplified lead system eliminates the sense/pace conductors required for pacing as this functionality is provided in the ICP 89. The umbilical wire 28 is used for communicating sense and pace events to the ICD.

Therapies and Modalities

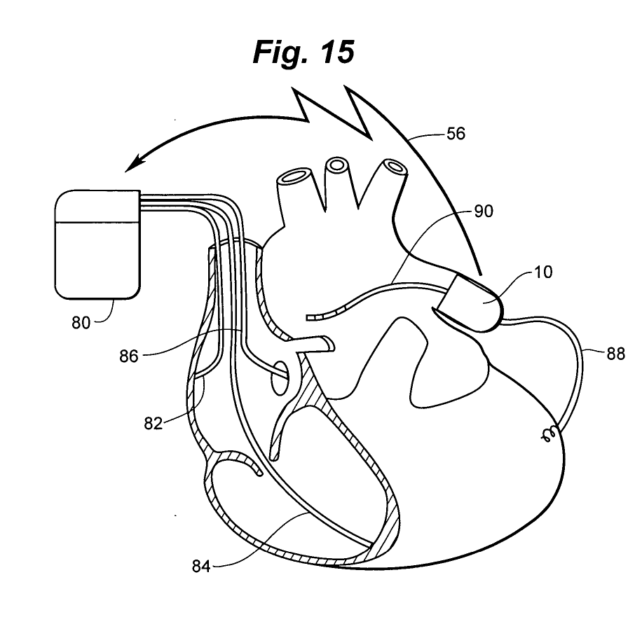

FIG. 15 is a relatively complex configuration that is intended to show the interaction between an ICP placed in the LAA and how this device can interact with the ICD or IPG 80. As seen in the figure the IPG/ICD 80 has a lead in the CS the RA and the RV. These leads are used for both sensing and pacing and if necessary defibrillation. The ICP 10 has two leads attached to it and one goes to the LA and the other to the LV. The preferred configuration for the ICP 10 device is placed in the LAA.

Access to the multiple signals in the atrium early in a heartbeat permits a better understanding of the atrial beat. Since this information is available early in the heart cycle it can be used to provide a therapy based upon a measurement of the quality of the atrial beat.

The device 10 may be in communication with an implanted ICD/IPG 80 through a RF link or a sub-threshold pulse train communication through the body with the ICD. In either event the atrial rhythm management device may inform the ICD that it is delivering a therapy and request that it not interpret the therapy as an episode of tachy arrhythmia. By the same token the atrial rhythm management device may invoke or activate the ICD and request synchronization of the defibrillation or cardioversion pulse with the pulse train delivered by the atrial rhythm management device ICP 10.

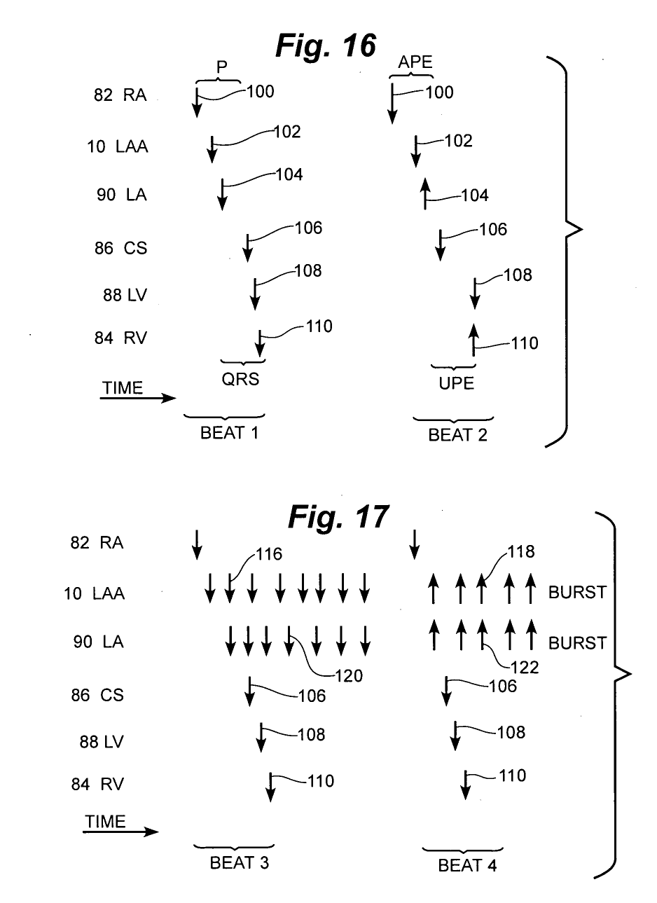

FIG. 16 is a timing chart that corresponds to the FIG. 15 configuration. The first beat seen in the figure is a naturally conduced beat of a healthy heart. The P-wave is first detected in the RA as indicated by sense event 100. Next the beat is conducted and sensed in the LAA as sense event 102. The beat next is sensed in the LA as sense event 104 that completes the electrographic P wave. After a brief A-V delay the signal is sensed in the coronary sinus (CS) as sense event 106. Next the depolarization is conducted through the apex of the LV and it is detected as sense event 108. The right heart contracts as well, initiated by the depolarization sensed as sense event 110. The observed activation sequence depends on the placement of the lead system and the specific anatomy of the LAA of the patient consequently, in many instances the LA electrode will sense a depolarization before the LAA lead.

Beat 2 corresponds to a wide QRS complex associated with CHF. In this beat the process is initiated by sense event 100 but the sense event 102 is “late” based on historical rhythm data. In this instance the ICP 10 initiates a pacing pulse to the LA as seen by pace event 112. Next a LV event detected by LV lead 88 gives rise to ventricular sense event 108. If the event occurs a the edge of its escape time a signal may be sent to the ICD/IPG 80 which may issue pacing pulse giving rise to pace event 114. In this fashion the ICP 10 attempts to “shorten” the duration of the p-wave and may invoke a pacing event from a remote device to shorten the QRS complex.

The device 10 may sense and process atrial electrograms and transmit those to either a remote device outside of the body, or another implanted device 80 which uses the event timing as part of a treatment choice. Due to its small size, the ICP 10 may enter a quiescent operating mode where it does not provide a therapy itself, but rather operates as a sensing and processing device for other more powerful implanted devices such as the ICD/IPG 80. The remote sensing capability based upon its placement high in the atrium can improve the ability to distinguish tachy arrhythmias.

FIG. 17 shows the interaction of the ICP 10 and the conventional IPG/ICD 80 in treating an episode of tachy arrhythmia. In beat 3 the lead 90 in the LA experiences a set of sense events that indicate a high atrial rate. This arrhythmia is not conducted as seen by the single sense event 106 in the CS confirmed by the single events in the LV and RV. If the treatment criteria are met then the ICP may treat the heart as set forth in beat 4. In the panel of beat 4 the ICD delivers stimuli typified by stimulus 118 to the LAA via the ICPs electrode. The stimuli are also sent to the LA lead 90 as typified by pace event 122. In this instance the ICP 10 may alert the ICD/IPG that an arrhythmia is taking place. Note that the electrodes available to the ICD/IPG 80 do not “see” the rhythm disturbance. In response to the alert the ICD/IPG may start charging its defibrillation capacitor to invoke a treatment is the arrhythmia is conducted to the ventricle.

What is claimed is:

1) An intracardiac pacer comprising:
a hermetic housing containing, a power source, a pacing circuit module;
a resilient deployable shield adapted to conform to said housing during insertion and deployable to an expanded shape that engages and anchors said housing in an anatomic location inside the heart.

2) The device of claim 1 wherein said shield is made from a Nitinol mesh.

3) The device of claim 1 wherein said shield is made from a Dacron mesh.

4) The device of claim 1 wherein said power source is a lithium solid state cell.

5) The device of claim 1 wherein said power source is a rechargeable battery.

6) The device of claim 1 further comprising;
an electrode site located at the distal tip of said housing for sensing and pacing heart tissue.

7) The device of claim 1 further comprising a lead system extending from said distal end of said housing adapted for placement in the heart.

8) A method of treating the heart comprising:
inserting an ICP into the LAA;
monitoring the atrial beat in the LAA;
setting a timing interval based on the sensed depolarization of the atrium based on the signal in the LAA;
programming the ICP to a pacing modality that supplies electrical energy to the LAA in response to a detected atrial beat measured in the LAA.

9) The method of claim 8 further comprising:
placing at least one electrode in a chamber selected from the group; LA, RA, LV, RV, and CS;
coupling said electrode to said ICP;
providing a pacing therapy from said ICP and said electrode.

10) The method of claim 8 further comprising:
a conventionally placed IPG coordinating its action with said ICP to provide dual chamber pacing therapy.

11) A method of treating a cardiac arrhythmia comprising the steps:
sensing an atrial depolarization from an electrode in the RA;
sensing the same depolarization from an electrode in LAA or RAA;
determine the conduction sequence and time interval between said measurements;
pacing a ventricle chamber if said measurement indicates a “wide” QRS.

12) A method of treating a cardiac arrhythmia comprising the steps:
sensing an atrial depolarization from an electrode in the RAA;
sensing the same depolarization from an electrode in LAA;
determine the conduction sequence and time interval between said measurements;
pacing the LA or LAA if said measurement indicates a “wide” atrial beat.

13) A method of treating a “wide QRS” cardiac arrhythmia comprising the steps:
sensing an atrial depolarization from an electrode in the RA;
sensing the same depolarization from an electrode in LAA;
determine the conduction sequence and time interval between said measurements;
pacing the LA or LAA if said measurement indicates a “wide” atrial beat and committed pacing of both LV and RV.
